(19) United States
(12) Patent Application Publication
(10) Pub. No.: US 2017/0086519 A1
(43) Pub. Date: Mar. 30, 2017

(54) GLOVES WITH SENSORS FOR MONITORING AND ANALYSIS OF POSITION, PRESSURE AND MOVEMENT

(71) Applicant: Sensoria, Inc., Redmond, WA (US)

(72) Inventors: Davide Giancarlo Vigano', Redmond, WA (US); Mario Esposito, Redmond, WA (US); Maurizio Macagno, Redmond, WA (US); Victoria Ann Esposito, Redmond, WA (US); Mauro Giusti, Redmond, WA (US); Francesco Pasquale Curra, Briar, WA (US)

(21) Appl. No.: 15/311,471

(22) PCT Filed: May 14, 2015
(86) PCT No.: PCT/US15/30899

§ 371 Date: Nov. 15, 2016

Related U.S. Application Data
(60) Provisional application No. 61/994,054, filed on May 15, 2014.

(51) Int. Cl. A41D 19/00; A61B 5/053; A61B 5/024; G09B 19/00; A61B 5/021; A61B 5/145; A61B 5/026; A61B 5/0205; A61B 5/00; A61B 5/08

(52) U.S. Cl. CPC A41D 19/0027; A61B 5/6806; A61B 5/0533; A61B 5/02438; A61B 5/0816; A61B 5/021; A61B 5/14542; A61B 5/026; A61B 5/0205; G09B 19/0038; A61B 2562/0247; A61B 2562/164; A61B 2560/0242; A61B 2562/0219

(57) ABSTRACT

Sensing devices including pliable e-textile pressure sensors are used in gloves intended to be worn against a user's hand, or a portion of a user's hand. Additional sensors, such as accelerometer(s), gyroscope(s) and geo-referencing sensor(s), may be incorporated in electronic devices that interface electronically with the pressure sensors and are mounted on or in proximity to a glove when in use. Systems and methods for storing, communicating, processing, analyzing and displaying data collected by sensor components for remote monitoring of conditions at hand surfaces, position and orientation data, movement data, and the like, are also disclosed. Sensors and sensor systems provide substantially real-time feedback relating to current body conditions, orientation and movement, and may provide notifications or alerts to users, coaches, etc., enabling early intervention when conditions indicate intervention is appropriate.

### FIELD

The present disclosure relates, generally to gloves having sensing and/or control features. In one aspect, the present disclosure relates to gloves incorporating pressure sensing systems. In another aspect, the disclosure relates to pressure sensing systems and enabling electronic devices and systems for detecting and monitoring hand position and gripping pressure relative to a handheld object and for detecting and tracking hand position and orientation during movement of the hand and the object.

### BACKGROUND

Various types of sensing systems have been incorporated in shoes, insoles, socks and garments for monitoring various physiological parameters for various applications, including recreational, sporting, military, diagnostic and medical applications. Medical applications for sensing pressure, temperature and the like for purposes of monitoring neuropathic and other degenerative conditions with the goal of alerting an individual and/or medical service providers to sensed parameters that may indicate the worsening of a condition, lack of healing, and the like, have been proposed. Footwear-related sensing systems directed to providing sensory data for patients suffering from neuropathy, for gait analysis, rehabilitation assessment, shoe research, design and fitting, orthotic design and fitting, and the like, have been proposed.

U.S. Pat. No. 8,925,392 discloses pressure sensors, interfaces and sensor systems for use in garments and with other types of flexible fabric and sheet-like substrates. PCT International Patent Application WO 2015/017712 discloses additional features of and applications for pressure sensors, interfaces and sensor systems, particularly for use in wearable apparel and medical applications.

Sensor-enabled gloves have also been proposed. U.S. Pat. No. 5,771,492 discloses an electronic golf glove training device, for example, that incorporates sensors in the form of mechanical, pressure sensitive switches located on a glove palm, thumb, finger or dorsal region. Electronic and sound-generating components are provided in a pocket located on the back of the glove and an audible signal is sounded when one of the switches is open, indicating a loss of pressure against one of the switches.

U.S. Pat. No. 5,733,201 discloses a golf-training glove having multi-function biofeedback instrumentation monitoring grip pressure and golf club head speed during a swing. Pneumatic pressure sensors are disclosed, and an instrumentation package is mounted on the back of the glove, displaying various data for the benefit of the user.

U.S. Pat. No. 7,780,541 discloses another golf training glove with sensors designed to trigger a signal when the user exerts excessive gripping pressure. Sensors are arranged on the glove fingers; electrical (especially capacitative) and pneumoelectrical sensors are disclosed as being suitable. Evaluation electronics are releasably connected to the glove by attachment to a base housing located at the top side of the glove.

U.S. Pat. No. 8,033,925 discloses a pressure sensing and notification system provided in a golf glove to detect excessive hand pressure during golf putting and swinging. The pressure sensor is a piezoresistive force sensor having an elongated flexible, printed circuit extending from a processor located on the back (top) of the glove with one or more pressure-sensitive ink sensors located at contact point(s). A vibrator signals the user when excessive pressure is detected.

U.S. Pat. No. 8,033,916 discloses a grip pressure sensor and swing monitor designed to be worn on the wrist to sense the flexing and relaxing of muscles and tendons of the user's wrist, which are said to reflect grip pressure. U.S. published patent application US 2010/0144455 discloses a wearable system for obtaining data relating to a player's golf game during swings and game play. Data collected by sensors located on or within a golf club or worn by a golfer are transmitted to a golf glove or a remotely located monitoring or display device. Electrical contact points provided on a glove may be used to detect hand positioning and grip during a swing.

U.S. Pat. No. 8,572,764 discloses an exercise glove having one or more sensors for detecting hand exercise events. Power, processing and memory features are also disclosed. U.S. published patent application US 2011/0302694 discloses a clinical sensing glove system to be worn by a clinician during administration of manual therapies for quantifying force, shear, hardness, and the like detected during the therapy.

### SUMMARY

In one aspect, sensor systems of the present invention comprise one or more sensor(s) mounted to/in/on or incorporated in or associated with (any of these arrangements being referred to as “associated with”) a flexible and/or pliable substrate that may be constructed as a (full or partial) glove, or another type of covering for the hand(s). The term “glove,” as we use it herein, refers to any type of covering for a hand, whether it covers the hand fully or partially. The term “sensor,” as we use it herein, refers to sensors as described herein, as well as means for sensing as that term may be construed to extend to sensors as described herein as well as other, additional types of sensors.

In one aspect, sensors associated with the substrate are capable of sensing a physiological parameter of the underlying skin or tissue; in another aspect, sensors are capable of sensing force or pressure exerted on or against the sensor by an underlying skin or tissue, and/or or by an object external to the body. Each sensor is electrically connected, (optionally) via one or more flexible leads, to a flexible conductive trace associated with the substrate, and conductive traces terminate at conductive signal transfer terminals associated with the substrate. Thus, sensor systems and sensing devices described herein preferably comprise at least one flexible sensor (or means for sensing), (optionally) at least two flexible leads, and at least two flexible traces associated with a pliable substrate that is or may be fashioned as a (full or partial) glove.

One or more of the sensor(s), flexible leads, and conductive traces is preferably stretchable and/or elastic, as well as being flexible and pliable, without diminishing the function or operating lifespan of the sensor, flexible lead or conductive trace. In some embodiments, the sensor(s), flexible leads and conductive traces may all comprise flexible, pliable electrically resistive and/or conductive fabric, thread or yarn materials. Gloves incorporating such sensor systems and sensing devices may be comfortably worn by users under many conditions, providing real time monitoring of conditions at or near body surfaces and reporting of the condition(s) to the user, a coach or teacher, colleagues, the general public or a selected audience, and/or a clinician.

The signal transfer terminal(s) on the substrate (e.g., glove) may be matingly received in signal receipt terminals associated with a Dedicated Electronic Device (DED) that, in some embodiments, is attachable to the substrate (e.g., glove) and serves as a (temporary or permanent) data collection device. The DED may also (optionally) house batteries or other energy storage devices and serve as a sensor charging device. The DED may have the capability of communicating with one or more external electronic device(s), such as a smartphone, personal computing device/display, host computer, centralized data processing system, or the like for signal transfer, processing, analysis and display to a user and/or others. In some embodiments, the external electronic device, and/or the DED, communicates with an external, hosted computing system (operated, e.g., at a centralized, hosted facility and/or in the “Cloud”) that provides additional data storage and/or processing and/or analysis, formulates feedback, notifications, alerts, and the like, that may be displayed to the user, a coach or teacher, colleagues, the general public or a selected audience, and/or a clinician, through one or more computing and/or display devices.

In some embodiments, one or more pressure sensor(s) associated with a substrate fashioned as a glove detect changes in voltage or resistance across a surface area that is associated with force exerted on the sensor, which is related to pressure (as force per unit surface area) and/or shear. In some embodiments, FSR (Force Sensitive Resistor) or piezo-resistive sensors may be used. One type of piezoresistive force sensor that has been used previously in footwear pressure sensing applications, known as FLEXIFORCE® sensors, can be made in a variety of shapes and sizes, and measure resistance, which is inversely proportional to applied force. These sensors use pressure sensitive inks with silver leads terminating in pins, with the pressure sensitive area and leads sandwiched between polyester film layers. FLEXIFORCE® sensors are available from Tekscan, Inc., 307 West First Street, South Boston, Mass. 02127-1309 USA. Other types of sensors may also be integrated in or associated with various substrate materials from which gloves are constructed (e.g., fabrics, leathers, sheet materials, and the like), including sensors providing data relating to temperature, moisture, humidity, stress, strain, galvanic skin response, heart rate, respiratory rate, blood pressure, blood oxygen saturation, blood flow, local gas content, bacterial content, multi-axis acceleration, orientation, positioning, and the like. Auxiliary sensors may also be associated with other substrates or auxiliary devices worn by a user, and data from such auxiliary sensors may be used with sensor systems as described herein to formulate feedback. Many suitable sensors are known in the art and may be adapted for use in sensing systems described herein.

In some embodiments, pressure sensors incorporated in sensing systems of the present invention comprise non-silicon-based materials such as pliable, piezo-resistive “e-textile” fabric material(s), threads or yarns, while associated leads and/or conductive traces comprise non-silicon-based materials such as pliable, conductive “e-textile” fabric materials, threads or yarns. In some embodiments, sensors and/or associated leads and/or conductive traces incorporated in sensing systems of the present invention comprise pliable, conductive fabric materials, threads or yarns that are substantially isotropic with respect to their flexibility and/or stretch properties. By “substantially” isotropic, we mean to include materials that have no more than a 15% variation and, in some embodiments, no more than a 10% variation in flexibility and/or stretch properties in any direction, or along any axis of the material. Suitable materials, such as piezo-resistive fabric sensors, coated and/or impregnated fabrics, including metallic coated fabric materials and fabric materials coated or impregnated with other types of conductive formulations, are known in the art and a variety of such e-textile materials may be used. In some embodiments, pressure sensors and conductive leads used in sensing systems described herein comprise the same pliable e-textile fabric materials having resistivity and conductivity properties, with resistive properties being used for sensing and conductive properties being used for signal transfer to conductive traces.

E-textile fabrics comprising a knitted nylon/spandex substrate coated with a resistive and/or conductive formulation are suitable for use, for example, in fabricating biometric pressure sensors and in other applications requiring environmental stability and conformability to irregular configurations. One advantage of using these types of e-textile fabric sensors is that they perform reliably in a wide variety of environments (e.g. under different temperature and moisture conditions), and they're generally flexible, durable, washable, and comfortably worn against the skin. Suitable flexible e-textile fabric materials are available, for example, from VTT/Shieldex Trading USA, 4502 Rt-31, Palmyra, N.Y. 14522, from Eeonyx Corporation, 750 Belmont Way, Pinole, Calif. 94564. E-textile fabrics are also being developed by Footfalls & Heartbeats Ltd., Ground Floor, Shed 20, 139 Quay Street, Princes Wharf, Auckland 1010, New Zealand.

Techniques for deriving force and/or pressure measurements using e-textile resistive and/or conductive fabric materials are known in the art and various techniques may be suitable. See, e.g., http://www.kobakant.at/DIY/?p=913. Techniques for measuring other parameters using e-textile fabric materials, such as humidity and temperature measurements, are also known and may be used in sensing systems of the present invention. See, e.g., http://www.nano-tera.ch/pdf/posters2012/TWIGS105.pdf. Moreover, techniques for detecting and measuring shear are also available. Fabric sensors of the present invention may thus be capable of monitoring various parameters, including force, pressure, shear, parameters derivative of or related to force, pressure and shear, humidity, temperature, gas content, and the like, at the site. Additional monitoring capabilities may be available using fabric sensors as innovation in fabric sensors proceeds and as nano-materials and materials incorporating nano-structures are developed and become commercially feasible. E-textile sensors capable of providing proportional pressure signals (e.g., proportional pressure sensed over a surface area), and/or providing pressure signals that correlate with spatial locations on a surface area of the e-textile sensors are preferred.

Flexible (and optionally stretchable or elastic) resistive or conductive e-textile fabric sensor(s), leads and/or traces are associated with a substrate such as fabric, (natural or synthetic) leather, or another material that's substantially non-conductive and pliable, is capable of being comfortably worn (directly or indirectly) against the skin, and is capable of moving in concert with the physiological structures the substrate is covering (e.g., fingers, thumbs and palms of the hand, and similar structures of other body areas). The term “fabric” or “sheet material” as used herein, refers to many types of pliable materials, including traditional (natural or synthetic) fabrics comprising woven or non-woven fibers or strands, as well as fiber reinforced sheet materials, and other types of flexible sheeting materials composed of natural and/or synthetic materials, including flexible natural and synthetic leather materials, various types of plastic sheeting material, pliable thermoplastic, foam and composite materials, screen-like or mesh materials, and the like. The substrate may comprise a sheet material fabricated from flexible material that is stretchy and/or elastic. The substrate may be substantially isotropic with respect to its flexibility and/or stretch properties. By “substantially” isotropic, we mean to include materials that have no more than a 15% variation and, in some embodiments, no more than a 10% variation in flexibility and/or stretch properties in any direction, or along any axis of the material.

For many glove and glove-like applications, one or more e-textile sensor(s) and/or sensing devices may be associated with a substrate that is in the form of a glove or a substrate that can be finished as a glove by sewing, bonding, weaving, or the like. The e-textile sensor(s) are positioned on inner, outer or intermediate location(s) with respect to the substrate surface for contacting portions of an individual's hand (e.g., finger(s), palm, etc.), directly or indirectly, during use and for detecting the position and/or magnitude of force and/or pressure exerted against the sensor(s) by an individual's hand, and by a handheld object. The sensor(s) may detect other parameters sensed at or near a skin surface. In some embodiments, conductive threads, yarns and/or e-textile fabric sensors may be sewn through or knitted into the substrate, sandwiched between two or more substrate layers, or otherwise incorporated in fabric, leather or other substrates. In some embodiments, e-textile fabric sensors may be partially or fully enclosed in a flexible barrier material or envelope to protect the sensor(s) from excessive moisture or other unfavorable ambient conditions.

The use of e-textile pressure sensors provided at selected predetermined locations on a glove provides precise identification of the position and grip pressure of a the user's hand with respect to a hand-held object, such as a golf club, another sports-related object (e.g., racket, bat, stick, etc.), a tool, machine or other occupational object, or the like. Feedback devices and analytics may be provided to alert the user to poor grip positioning of the object in the hand, inappropriate grip pressures exerted (statically or dynamically, during motion of the hand and object), and the like. Similarly, sensor-enabled gloves used for weightlifting, lifting or pushing objects, or the like, may provide precise data relating to force exerted against specific sensor locations. The use of e-textile pressure sensors provided at selected predetermined locations on a glove may also provide precise data relating to the location and magnitude of impact forces exerted against the sensors and sensor location on a hand (in a boxing glove, for example).

Each sensor, such as a resistive pressure sensor, may be associated with two conductive leads, and each of the leads may be electrically connected to a conductive trace conveying electrical signals to a signal transfer terminal. In some embodiments, resistive pressure sensors may be directly connected to conductive traces. Resistive e-textile fabric sensors, as previously described, may be electrically connected to conductive leads, or may have a flexible fabric lead associated with or incorporated in (e.g., formed as part of) the fabric sensor footprint. The sensors and/or conductive leads are electrically connected to flexible conductive traces, which may comprise a variety of flexible conductive materials, such as a conductive fabric, thread, yarn, or the like. In some embodiments, the conductive traces are stretchable and/or elastic, at least along the longitudinal axis of the conductive trace. In some embodiments, conductive traces comprise a conductive e-textile fabric, thread or yarn having high electrical conductivity, such as silver coated e-textile materials, and may be sewn to or woven through the underlying substrate material, or bonded to the underlying substrate material using adhesives, heat bonding or non conductive threads. Suitable conductive fabric, thread and yarn materials are known in the art and are available, for example, from the vendors identified above.

Each of the conductive traces terminates in a signal transfer terminal that is mounted to/in/on, or associated with (collectively referred to as “associated with”), the substrate and can be interfaced with a mating signal receipt terminal of a dedicated electronic device (DED) providing energy, data storage, processing and/or analysis capabilities. In general, conductive traces and signal transfer terminals are arranged in a predetermined arrangement that corresponds to the arrangement of signal receipt terminals in the DED. Many different types of signal transfer and receipt terminals are known and may be used in this application. In one exemplary embodiment, signal transfer and receipt terminals may be mounted in cooperating fixtures for sliding engagement of the terminals. In another embodiment, signal transfer terminals may be provided as conductive fixtures that are electrically connected to the conductive trace (and thereby to a corresponding sensor) and detachably connectible to a mating conductive fixture located on the DED. The mating terminals may comprise mechanically mating, electrically conductive members such as snaps or other types of fasteners providing secure mechanical mating and high integrity, high reliability transfer of signals and/or data. In some embodiments, convenient and secure mating of the terminals may be enhanced using magnetic mechanisms or other types of mechanisms that help users to properly connect/disconnect the mating terminals with minimal effort.

The DED, in addition to having data recording, processing and/or analysis capabilities, may incorporate an energy source such as a battery providing energy for data recording, processing and/or analysis, as well as providing energy for operation of one or more of the sensor(s). The energy source is preferably a rechargeable and/or replaceable battery source. The DED generally provides a lightweight and water-tight enclosure for the data collection and processing electronics and (optional) energy source and provides signal receiving terminals that mate with the signal transfer terminals communicating with the sensor(s) for conveying data from the sensors to the DED.

DEDs having signal receipt terminals that mate with the signal transfer terminals associated with the substrate may take a variety of form factors, depending on the form factor of the underlying sensing substrate and/or the conditions and location of the device during use. When sensors are incorporated in a glove or glove-like form factor for monitoring conditions sensed at the hand, for example, the signal transfer terminals may be arranged in proximity to one another at a back of the hand location on the glove, and the DED may have a form factor of a small dongle or tab, with mating signal receiving terminals sized and configured for mating with the underlying signal transfer terminals. In one embodiment, the signal transfer terminals and (detachable) DED may be located on the back of the glove in a wrist area in proximity to or below an adjustment tab. In another embodiment, the signal transfer terminals (and detachable DED) may be located on an adjustment ta, such as on a flap that adjustably tightens the glove.

The DED may be provided as a substantially flexible or a substantially rigid component, and it may take a variety of forms. In one embodiment, the DED may comprise a housing that can be positioned in a pocket provided on a surface of the glove that provides secure positioning of the DED during movement, while allowing removal of the DED for charging and other purposes. In another embodiment, the DED may comprise a housing that mechanically mates with a base fixed or attachable to the surface of the glove and providing access to the signal transfer terminals on the glove. Various alternative systems for securely mounting the DED on an underlying glove and for connecting the signal transfer and signal receipt terminals may be provided.

The DED preferably communicates with and transfers data to one or more external computing and/or display system(s), such as a smartphone, computer, tablet computer, dedicated computing device, or the like, using wired and/or wireless data communication means and protocols. The DED and/or an external computing and/or display system may, in turn, communicate with a centralized host computing system (located, e.g., in the Cloud), where further data processing and analysis takes place. Substantially real-time feedback, including data displays, notifications, alerts and the like, may be provided to the user, coach, or the like. In some embodiments, the DED may store the data temporarily to a local memory, and periodically transfer the data (e.g., in batches) to the above mentioned external computing and/or display system(s). Offline processing and feedback, including data displays, notifications and the like may be provided to the user, caretaker, and/or clinician according to user, caretaker and/or clinician preferences.

Additional sensors and electronics may be provided in gloves and/or in DEDs as described herein. In some embodiments, for example, one or more accelerometer(s) (e.g., single and/or multi-axis accelerometer(s)) and/or gyroscope(s) may be provided for movement and/or orientation sensing in addition to the sensor(s) for detecting hand position relative to an object, force and/or pressure sensing. Sensors that detect physiological conditions such as stress, sweat, moisture, conductivity, and the like, may also be provided. Various types of location (e.g., GPS), optical and/or audio detectors (e.g., microphone(s)) and sensors may also be used. These electronic devices are typically located in the DED, where power, memory, processing and/or analytical capabilities are available. Additional sensors and electronic devices may also be provided.

In some embodiments, for example, a gyroscope or another orientation sensor may be provided in a DED or sensor-enabled glove to provide data relating to hand (and object) orientation, statically and/or dynamically during movement of the hand (and object). Orientation data is valuable, for example, in sporting applications such as golf, when the ball is addressed prior to swinging. The “correct” orientation of the hands and golf club grip when the ball is addressed during putting, for example, is an upright, vertical orientation of the stem and grip of the golf club. The “correct” orientation of the hands and golf club grip when the ball is addressed during driving is an entirely different orientation. Real-time feedback provided to the user from sensing systems/DED/analytical facilities described herein may present data and corrective feedback to the user, e.g., by alerting the user when his or her hand/grip position is out of alignment, improving the user's play and reducing injuries. Grip pressure and the location and distribution of grip pressure during play and ball striking is also important. Orientation and grip pressure sensing of other sporting implements (various types of rackets (e.g., tennis, racket ball, squash, and the like), bats (e.g., baseball and cricket), sticks, (e.g., hockey and lacrosse), is also important and may also be detected using sensing systems as described herein.

In some embodiments, one or more accelerometer(s) may also collect data relating to various aspects of motion of the hand(s) and object during movement. Incorporation of a gyroscope and/or accelerometer in a glove or DED associated with a glove may also provide data relating to the 3D motion of the hands (and club) during the swing. In one embodiment, a 3D representation of the plan of the swing, as detected by the position and movement of the golfer's hand(s) and the golf club grip may be detected and displayed. In yet additional embodiments using location sensors, geo-referencing tools, and other sensors (e.g., audio), swings and/or ball strikes may be detected and mapped (e.g., on specific golf courses), providing the user with a stroke count, precise information relating to the number of swings taken, swing (and ball) locations, etc. Data collected may be stored, reviewed by the user, or a coach, compared to the user's data or to desired “standards,” data collected from other users, etc.

In operation, an initial authentication routine and/or user identification system matches the DED and associated sensing system (e.g., the collection of sensor(s) associated with an underlying substrate) with the user, coach or the like, and may link user information or data from other sources to a software- and/or firmware-implemented system residing on the external computing system. The external computing device may itself communicate with a centralized host computing system or facility where data is stored, processed, analyzed, and the like, and where output, communications, instructions, commands, and the like may be formulated for delivery back to the user, coach, or the like, through the external computing device and/or the DED.

Calibration routines may be provided to ensure that the DED and connected related sensor system are properly configured to work optimally for the specific user. Configuration and setup routines may be provided to guide the user to input user information or data to facilitate data collection, and various protocols, routines, data analysis and/or display characteristics, and the like, may be selected by the user to provide data collection and analysis that is targeted to the specific user. Notification and alarm systems may be provided, and selectively enabled, to provide messages, warnings, alarms, and the like to the user, substantially in real-time, based on sensed data.

The use of similar types of sensors and sensing systems in garments such as socks and in association with substrates such as bandages, sheets and other flexible substrates, is described in detail in commonly owned U.S. Pat. No. 8,925,392 and in PCT Patent Publication WO2015/017712 A1, which are incorporated herein by reference in their entireties.

### BRIEF DESCRIPTION OF THE DRAWINGS

FIGS. 1A, 1B and 1C show schematic illustrations of exemplary e-textile sensors having different sensor geometries and leads positioned in different locations and configurations.

FIGS. 2A and 2B illustrate exemplary sensor contact locations on the palms of the hand for positioning of position and/or pressure sensors associated with a golf glove to evaluate hand positioning and grip pressure during golf swings; FIG. 2C illustrates placement of position and/or pressure sensors in locations on the palm of a golf glove for monitoring the contact locations shown in FIG. 7B.

FIGS. 3A, 3B and 3C schematically illustrate the handle of a golf club contacting different areas of the palm and fingers and contacting, or not contacting different spatial regions of selected position and/or pressure sensors.

FIGS. 4A and 4B show schematic diagrams illustrating two glove substrates that, when joined at the peripheral edges, form a glove. Exemplary sensors, leads, traces and signal transmitting terminals are illustrated. FIG. 4A illustrates three pressure sensors positioned on the palmar portion of the glove; FIG. 4B illustrates two pressure sensors positioned on the palmer portion of the glove and one pressure sensor positioned on a finger portion of the glove.

FIG. 5 shows a schematic diagram illustrating a boxing glove having pressure sensors located to detect pressure and impact force exerted against predetermined locations of the glove.

FIG. 6 shows a block diagram illustrating basic components of an exemplary data collection device (e.g., DED), illustrating its interface with sensors associated with a substrate, an external computing device, and a centralized host system maintained, for example, in the Cloud.

FIG. 7 illustrates one exemplary layout of another exemplary data collection device (e.g., DED), illustrating its interface with sensors associated with a substrate, additional sensors located on the data collection device, processing and communications features.

FIGS. 8A-8B schematically illustrate one embodiment of a DED that detachably mounts to a glove. FIG. 8A shows an exemplary location for mounting a DED in a dedicated pocket on the back of a glove; FIG. 8B shows an exemplary schematic diagram illustrating the bottom of a DED having a plurality of contacts and the top of the DED having indicators. FIG. 8C shows the exemplary diagram of FIG. 8B additionally illustrating, in a highly schematic format, a display component associated with the glove.

FIG. 9 illustrates another exemplary embodiment of a housing that can be mounted at a desired location on a glove for interfacing with a mating DED having signal receiving terminals and electronics.

FIG. 10 illustrates an exemplary graphical user interface and application screen illustrating graphics and exemplary information displayed in a golf glove application.

It will be understood that the appended drawings are not necessarily to scale, and that they present simplified, schematic views of many aspects of systems and components of the present invention. Specific design features, including dimensions, orientations, locations and configurations of various illustrated components may be modified, for example, for use in various intended applications and environments.

### DETAILED DESCRIPTION OF EXEMPLARY EMBODIMENTS

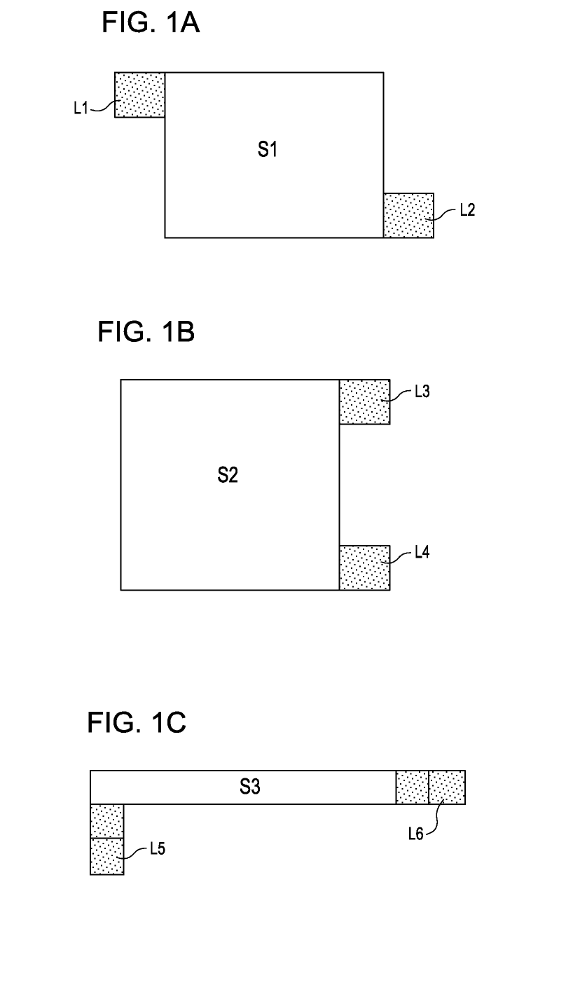

FIG. 1A shows an exemplary fabric sensor S1 with leads L1 and L2. In this example, sensor S1 comprises a generally rectangular piece of e-textile conductive and/or resistive fabric, and conductive leads L1 and L2 are positioned extending from opposite sides of sensor S1. Conductive leads L1 and L2 may be provided as integral extensions, or pieces, of the same conductive and/or resistive fabric of sensor S1. Alternatively, leads L1, L2 may comprise a material having different properties than those of the sensor, and leads L1 and L2 may be operatively and electrically connected to the sensor so that sensor signals are reliably and effectively transmitted through leads L1 and L2.

FIG. 1B shows a similar type of fabric sensor S2 having leads L3, L4 extending from a common side of the sensor. As described with reference to FIG. 1A, conductive leads L3, L4 may be provided as integral extensions, or pieces, of the same conductive and/or resistive fabric of sensor S2, or leads L3, L4 may comprise a material having different properties than those of the sensor, and may be operatively and electrically connected to the sensor so that sensor signals are reliably and effectively transmitted through leads L3 and L4.

FIG. 1C shows a similar type of fabric sensor S3 having a generally L-shaped configuration, with leads L5, L6 extending from terminal ends of the L-shaped sensor. As described with reference to FIGS. 1A and 1B, conductive leads L5, L6 may be provided as integral extensions, or pieces, of the same conductive and/or resistive fabric of sensor S3, or leads L5, L6 may comprise a material having different properties than those of the sensor, and may be operatively and electrically connected to the sensor so that sensor signals are reliably and effectively transmitted. In the exemplary embodiment illustrated in FIG. 1C, sensor S3 has a generally rectangular configuration and has a length at least twice the length (or corresponding dimension) of leads L5, L5; in some embodiments, sensor S3 has a length of from about 2 times to about 6 times the length (or corresponding dimension) of leads L5, L6.

E-textile fabric sensors are mounted to, or associated with, an underlying substrate (e.g., natural or synthetic leather, a fabric or sheet material, or the like) in a variety of ways, including sewing (e.g., stitching), adhesive bonding, thermal bonding, and the like. It will be appreciated that although rectangular and L-shaped polygonal sensor/lead combinations are illustrated, fabric sensors having a variety of sizes and configurations may be provided. Leads having the same properties as the sensors may be used, or other types of leads may be employed. It will also be appreciated that the arrangement of leads with respect to sensor(s) may vary, depending on the properties, size and configuration of the sensor and lead components and that, in some embodiments, sensors may be connected directly to traces without using designated leads.

In some embodiments, one or more protective layers or materials may be provided to protect one or more sensor(s) and, optionally, the associated leads, and all or portions of conductive traces, from contact with liquids, body fluids or other materials present in the ambient environment (e.g., moisture, rain, etc.), while preserving the core resistive features and functions of the sensor(s). A protective barrier may comprise a liquid impervious or substantially liquid impervious material, such as a generally thin plastic sheet material or a composite sheet material, that doesn't interfere with the sensing capacity of the sensor. By “substantially” liquid impervious we mean that liquid penetration of the material is insubstantial enough to affect the features and functions of the sensor(s). The protective barrier may optionally be breathable and/or gas permeable. Many such liquid impervious barrier materials are known. In some embodiments, a protective barrier may be provided on one surface of the sensor; in some embodiments, a sandwich- or envelope-type barrier that substantially seals the sensor in a substantially liquid impermeable envelope or pouch may be used.

E-textile sensors as disclosed herein are electrically connected to conductive traces, as described. The conductive traces terminate in conductive terminals, which are typically associated with the substrate (e.g., glove) and provide connectivity to mating conductive terminals provided in a DED. In many applications, sensors, leads and/or traces may be associated with an interior surface of a substrate and conductive terminals CT1, CT2 may be provided as conductive elements, such as conductive mechanical snaps, that penetrate the substrate material from the interior to the exterior substrate surface. In some embodiments, signal transfer and signal receipt terminals have mechanically and magnetically detachable mating capabilities. The polarity of magnetic components may be arranged to provide male and female connectors that are connectable only when magnetically aligned in a predetermined orientation, which may facilitate user connection of the mating terminals in an appropriate orientation. In this exemplary embodiment, properties of the magnetic field may be used to create terminals that can only connect in one orientation: in this way, the user is guided to properly connect signal receiving terminals provided on the DED to the sensor system(s) associated with the underlying substrate.

It will be appreciated that other types of sensors may be integrated in the sensor system that contact the underlying body area (directly or indirectly), and that additional conductive terminals may be provided for transmission of signals and/or data from other types of sensors. It will also be appreciated that multiplexing systems may be implemented and that multiple sensors and sensor traces may be associated with a single conductive terminal. In some embodiments, the mating and detachment of terminal interfaces on the substrate and DED may operate as switches that are switched on and off abruptly by an external driving force from one switch position (e.g., “activated” when the terminals are connected) to a second position (e.g., “off,” when the terminals are detached).

Sensing systems, as disclosed herein, may be used in connection with various types of substrates and garments, as previously described. In specific embodiments disclosed herein, such sensing systems are embodied in gloves and other close-fitting articles configured to be worn on a hand, or on part of a hand. Sensor-enabled gloves may be used and designed specifically for various applications in which an object is gripped to provide data relating to hand position and/or grip pressure on the object, in both static and dynamic settings. This capability enables a user wearing a sensor-enabled glove as described herein to analyze his or her grip on the object before, during and/or after movement of the object.

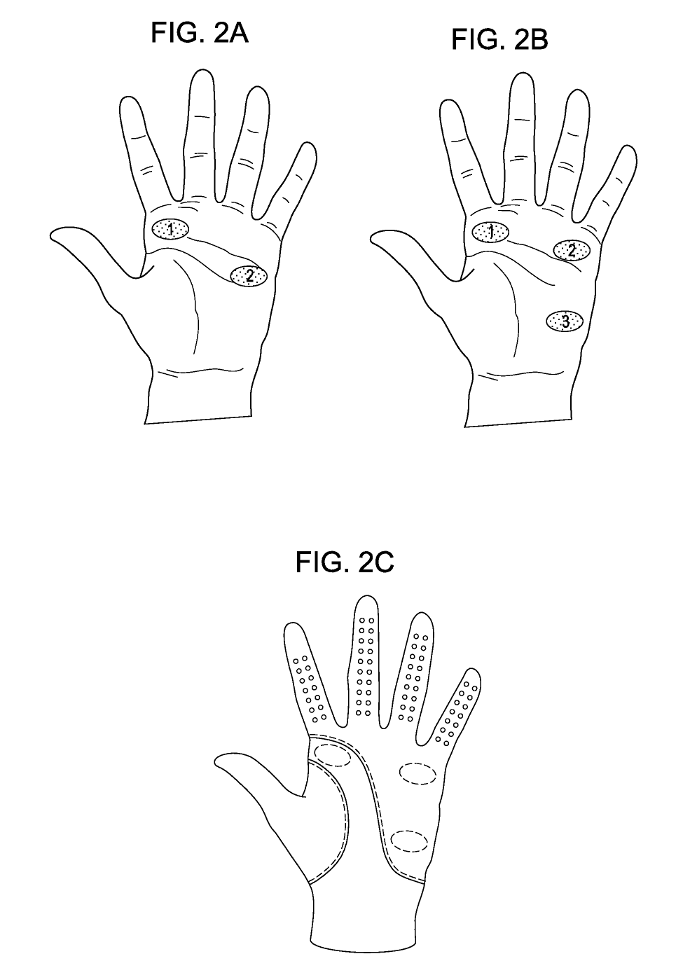

FIGS. 2A-2C illustrate regions on a palm suitable for sensor placement for purposes of monitoring golf grips and golf swings, and positioning of sensors on a glove intended to be used for golfing. In the two sensor embodiment schematically illustrated in FIG. 2A, an e-textile sensor as described herein is positioned to contact the metacarpophalangeal joint (MCP) II (sensor 1 shown in FIG. 2A), and another e-textile sensor as described herein is positioned below the metacarpophalangeal joint (MCP) V (sensor 2 shown in FIG. 2A). In a three sensor embodiment schematically illustrated in FIG. 2B, an e-textile sensor as described herein is positioned to contact the metacarpophalangeal joint (MCP) II (sensor 1 shown in FIG. 2B), another e-textile sensor as described herein is positioned to contact the metacarpophalangeal joint (MCP) V (sensor 2 shown in FIG. 2B), and a third e-textile sensor as described herein is positioned to contact the outer palm in an area in proximity to the middle of the metacarpal bone V (sensor 3 shown in FIG. 2B). FIG. 2C shows the location of sensors 1, 2 and 3, positioned as described above, on inner surfaces of a glove.

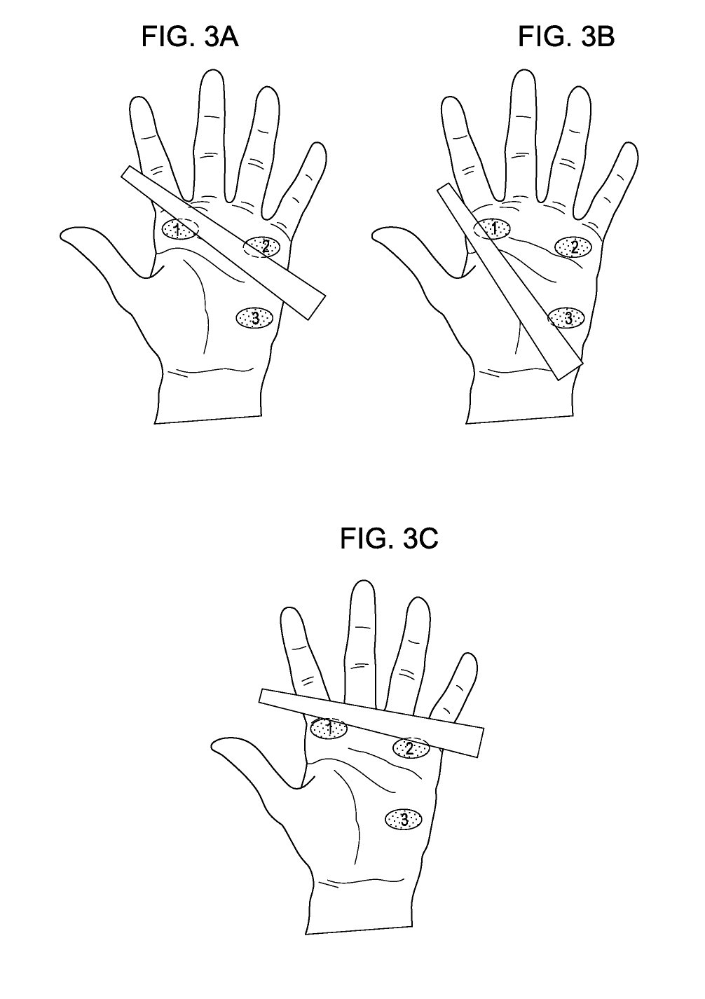

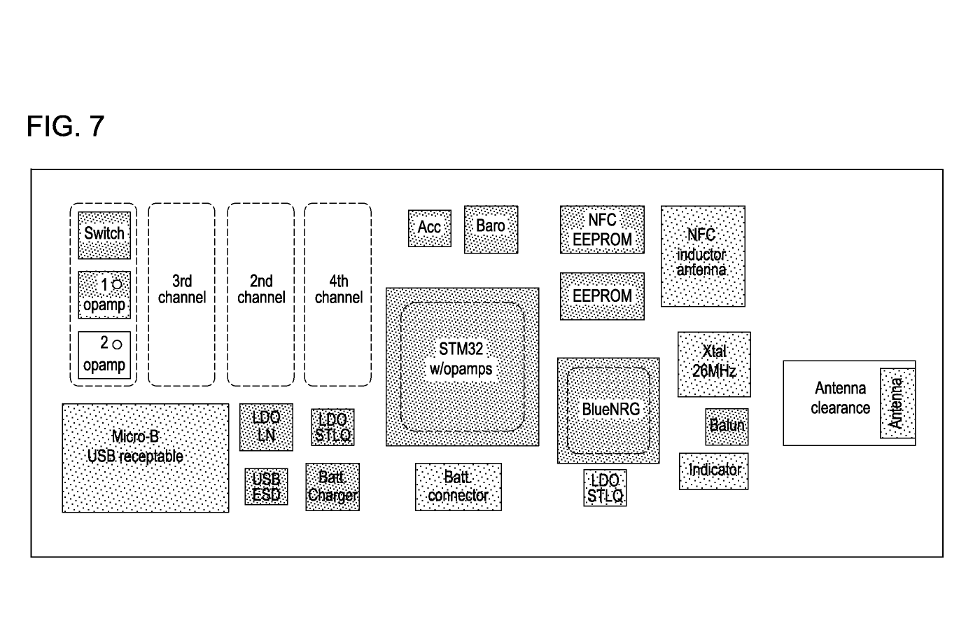

FIGS. 3A-3C schematically illustrate various golf club grip patterns in a user's hand with reference to the sensor locations. FIG. 3A illustrates a “good” grip, where the golf club is gripped at an angle between the MCP II (sensor 1) and MCP V (sensor 2) and rests above metacarpal bone V (sensor 3). This “good” grip is characterized by contact and pressure of the golf club grip on medial portions of sensors 1 and 2, and by the absence of contact and pressure on sensor 3. FIG. 8B illustrates a “bad” grip, where the golf club is gripped predominantly in the palm of the hand. This “bad” grip is characterized by contact and pressure of the golf club grip in the lateral region of sensor 1, the absence of contact and pressure on sensor 2, and slight contact and pressure on a medial portion of sensor 3. FIG. 8C illustrates another “bad” grip, where the golf club is gripped predominantly at the base of the finger region of the hand. This “bad” grip is characterized by contact and pressure of the golf club grip in the distal regions of sensors 1 and 2, and the absence of contact and pressure on sensor 3.

These examples show how using position and grip pressure sensors in these locations, in combination with a DED and/or external data processing and analytical facility and data analysis, provides detection of golf club grip placement and grip pressure in the user's hand. Data relating to the grip placement and pressure may be detected statically, at one or more time points, and may also be detected and monitored dynamically over periods of time, such as during golf swings, for tracking, in real-time, how the grip placement and grip pressure varies during golf swings. This information may be used by glove wearers, coaches, medical professionals, colleagues and the like, to improve golf club hand positioning and grip pressure, reduce injuries, etc.

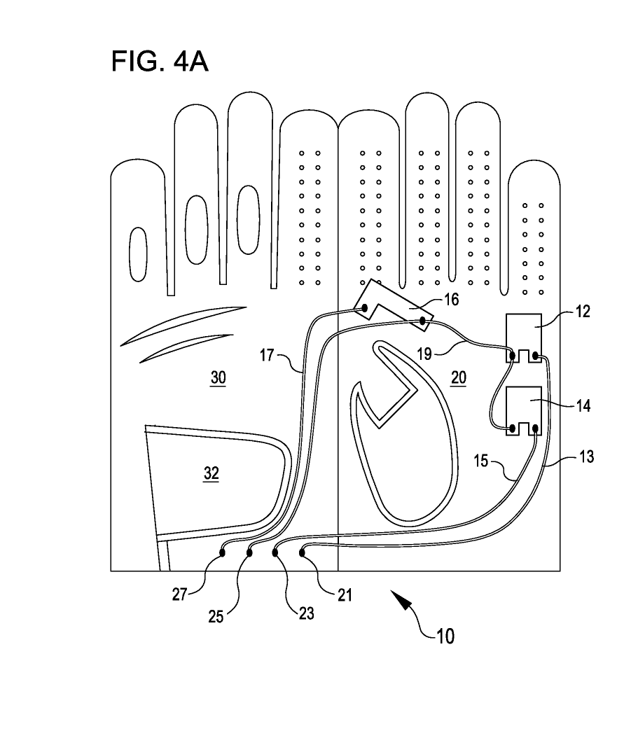

FIG. 4A schematically illustrates one exemplary layout for a glove having three discrete position and pressure sensors located on the palm region of the glove. Specific sensor, lead, trace and terminal configurations, orientations and positioning are illustrated; it will be appreciated that numerous other sensor, lead, trace and terminal configurations, orientations and positions may be employed, particularly for use in different glove and sporting applications. In the embodiment illustrated in FIG. 3A, generally rectangular resistive pressure sensors 12, 14 having leads extending from a common side (such as the sensor/lead configuration illustrated in FIG. 1B) are positioned at an interior surface of the glove substrate 10 (and directly or indirectly contacting the glove surface) at locations corresponding to sensor positions 2 and 3 as shown above with reference to FIGS. 2B and 2C. These sensor locations correspond (at least generally) to the metacarpophalangeal joint (MCP) V (sensor 2 shown in FIG. 2B), and the outer palm in an area in proximity to the middle of the metacarpal bone V (sensor 3 in FIG. 2B). A generally L-shaped resistive pressure sensor 16 is positioned at the base of the index finger in proximity to the metacarpophalangeal (MCP) joint II. The L-shaped sensor has a longer leg extending from a location corresponding to the base of the index finger toward MCP V (sensor 2) and oriented at an angle of from about 10° to about 70° from horizontal. The shorter leg of the L-shaped sensor extends toward a lateral edge of the glove and is oriented at an angle of from about 10° to about 70° from vertical.

Leads may be provided integrally with and as extensions from sensors 12, 14 and 16 as shown in FIG. 4A. The leads for sensors 12 and 14 extend proximally from generally rectangular sensors, and the leads of the L-shaped sensor 16 are formed at the ends of each extending leg. One suitable arrangement of traces is illustrated in FIG. 4A. In this embodiment, a dedicated trace 13, 15 and 17 is electrically connected to one lead of each sensor and terminates at a dedicated signal transfer terminal 21, 23, 27, respectively, as shown. Signal transfer terminals mate and communicate with signal receipt terminals in a DED and may provide signals to discrete channels for processing. The signal transfer terminals, as shown in FIG. 4A, are provided on one face (the outside or upwardly facing surface) of the glove, while the sensors are provided on the palmer face of the glove.

Another lead from each sensor (12, 14, 16) is connected, serially, to a common trace 19, which terminates in a dedicated signal transfer terminal 25. This common trace may serve as a ground trace, terminating in a ground terminal. Alternatively, these sensor leads may be connected in parallel to a common ground terminal. It will be appreciated that many different conductive trace arrangements may be used for both signal traces and ground traces.

In some embodiments in which the traces are stitched through the substrate material of the glove, the location and configuration of contrasting trace stitching may correspond to desired positioning of an object (e.g., golf club grip) across the palm, providing a visual guide to the wearer of proper hand and club positioning.

The sensor/lead components may be associated with a palmer component 20 of the glove, as shown, and the signal transfer terminals are associated with an outer or dorsal component 30 of the glove. In the embodiment illustrated in FIG. 4A, the signal transfer terminals are arranged in a linear configuration near the base of the dorsal component of the glove or in the wrist region of the glove. In alternative embodiments, signal transfer terminals may be arranged on a glove adjustment tab 32 or elsewhere on the (outer) dorsal side of the glove. In yet other embodiments, the signal transfer terminals may be arranged on a palmer side 20 of the glove at a base or wrist region.

Different configurations and locations of e-textile sensors may be provided in gloves for different applications. FIG. 4B illustrates another embodiment of a sensor-enabled glove in which resistive pressure sensors 42, 44 having leads extending from a common side (such as the sensor/lead configuration illustrated in FIG. 1B) are positioned at an interior surface of the glove substrate 50 (and directly or indirectly contacting the glove surface) at locations corresponding to sensor positions 2 and 3 as shown above with reference to FIGS. 2B and 2C. An additional resistive pressure sensor 46 is positioned at an interior surface of a glove finger, shown as the index finger in FIG. 4B. In additional embodiments, resistive pressure sensors may be positioned at different locations on the palmer area of the glove, and also at different or additional finger regions.

Leads may be provided integrally with and as extensions from sensors 42, 44 and 46 as shown in FIG. 4B. Leads for exemplary sensors 42, 44 and 46 may extend proximally from generally rectangular sensors, as shown. In the embodiment illustrated in FIG. 4B, dedicated signal traces 43, 45 and 47 are electrically connected to one lead of each sensor 42, 44 and 46, respectively, and terminate at dedicated signal transfer terminals 51, 53, 57, respectively, as shown. Signal transfer terminals mate and communicate with signal receipt terminals in a DED and may provide signals to discrete channels for processing. The signal transfer terminals, as shown in FIG. 4B, are provided on one face (the outside or upwardly facing surface) of the glove, while the sensors are provided on the palmer face of the glove. Another lead from each sensor (42, 44, 46) is connected, serially, to a common trace 49, which terminates in a dedicated terminal 55. This common trace 49 may serve as a ground trace, and terminal 55 may serve as a ground terminal. In some embodiments, sensors may be connected in parallel to a common ground terminal, and many different conductive trace arrangements and configurations may be used for both signal traces and ground traces.

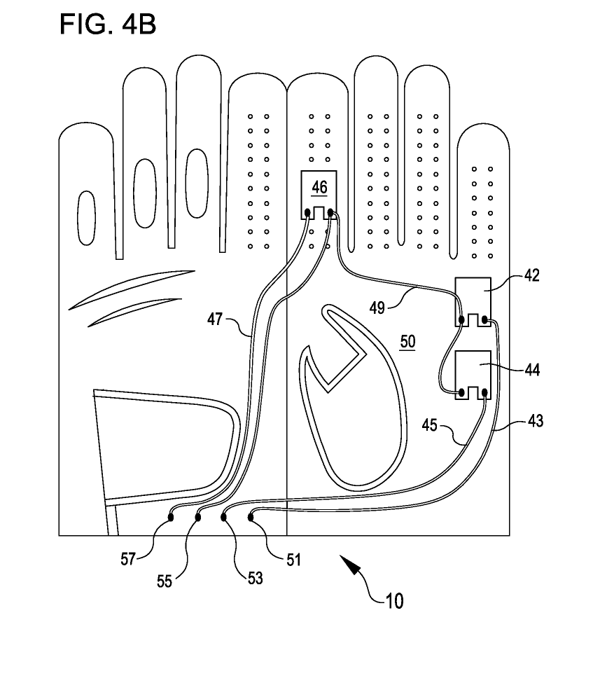

FIG. 5 illustrates a highly schematic view of a boxing glove 52 incorporating a plurality of resistive pressure sensors 54, 56, 58, shown positioned internally of the external surface of glove 52 and on a distal portion of the glove. Resistive pressure sensors, as disclosed herein, may be used to detect and report pressure exerted on or by the glove at the sensor locations, and to detect and report impact forces experienced at the sensor locations. Sensors may be provided at different or additional locations, including at a position corresponding to the heel portion of the hand, along the outer or medially-facing portion of the glove, at or near a thumb portion of the glove, or the like.

A dedicated electronic device (DED) has signal receipt terminals that mate with conductive signal transfer terminals to provide signal and/or data transfer from the sensor/lead/traces associated with the substrate to the DED. The DED typically comprises a protective housing or case enclosing an interior space containing processing, memory and/or communications components. The DED preferably includes a protective and watertight housing or case protecting the electronic components provided within the housing. The housing may be provided as a substantially rigid or a substantially flexible component and a variety of DED form factors may be provided, depending on the type and arrangement of underlying substrate and signal transfer terminals.

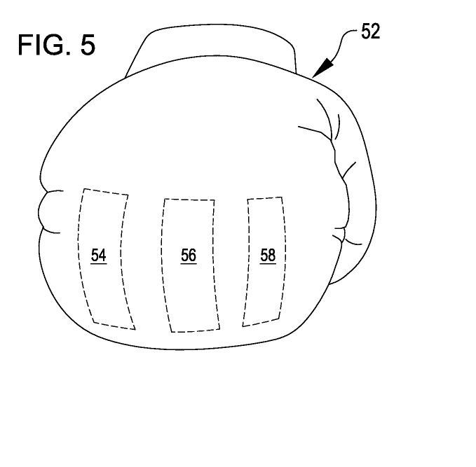

The DED incorporates processing, memory and/or communications functionalities within the housing. A schematic diagram illustrating exemplary DED components and interfaces is shown in FIG. 6. The DED has signal receipt terminals (shown as “connectors”) that feed analog input signals to appropriate processing devices, such as analog filters, A/D converters, and to a processor. Optional manual control input(s) and one or more optional output display(s) may be provided in or on the DED, as shown. Local memory may also be provided, and means for communicating signals and/or data externally via wired or wireless protocols may be provided, as shown. Circuitry in the DED may be provided for reading the sensor signals; firmware may be provided for processing signal data, applying post processing algorithms and formatting the data for communication to an external computing and/or display device. The DED may incorporate firmware and/or software components for collecting, filtering, processing, analyzing data, or the like. In one embodiment, the DED hosts firmware subroutines that apply at least some of the following: low pass filtering algorithms to reduce incoming signal noise; pull up resistors logic to avoid shorting of the device and additional noise filtering.

Signals and/or data are communicated from the DED to an external computing facility or device, such as a computer, base station, smartphone, or another bridge device, and/or to a centralized, hosted facility in a remote location, such as in the Cloud or at a centralized data processing and analysis facility. Following data analysis in accordance with predetermined and/or pre-programmed instructions, data output, analysis, notifications, alerts, and the like are communicated from the centralized hosted facility to the bridge device, and/or the DED, as shown. It will be appreciated that this is one exemplary data flow scheme, and that many other work flows may be advantageously used in connection with sensing systems of the present invention.

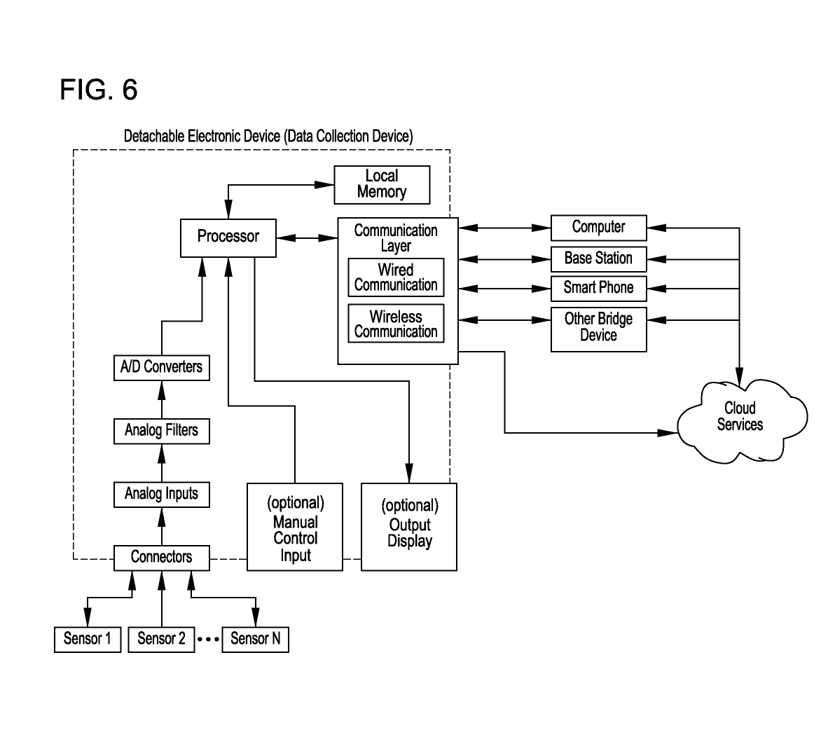

FIG. 7 schematically illustrates an exemplary 4-channel layout of another DED component. Three dedicated channels (2^{nd}, 3^{rd}, 4^{th}) are provided for signal receipt from three sensor/trace combinations, and another channel including an activation switch serves as the common signal receipt terminal communicating with each of the three sensors via a common (serial) trace, or via parallel traces. This exemplary DED comprises additional sensor components, including an accelerometer (Acc) and barometer (“Baro”), as well as a battery connector, battery charger, micro-USB port, processor, memory components, antennae and communications components, as shown.

In some embodiments, the DED may additionally comprise visual indicators or display elements for providing feedback to a user. Additionally or alternatively, signals and/or data may be communicated from the DED to an external computing facility or device, such as a computer, base station, smartphone, or another bridge device, and/or to a centralized, hosted facility in a remote location, such as in the Cloud or at a centralized data processing and analysis facility. Following data analysis in accordance with predetermined and/or pre-programmed instructions, data output, analysis, notifications, alerts, and the like may be communicated from the centralized hosted facility to the bridge device, and/or the DED, as shown in FIG. 6. It will be appreciated that one exemplary data flow scheme has been described and illustrated, and that many other work flows may be advantageously used in connection with sensing systems of the present invention.

In one embodiment, the DED may be physically attached to the sensing substrate (e.g., a glove) for data collection and then detached from the sensor terminals and physically mounted (e.g., though a USB or another wired connection), to an external computing and/or display device such as a phone, personal computing device, computer, or the like to download data. In other embodiments, the DED preferably has wireless communication capability (e.g., using Bluetooth, WiFi, or another wireless standard) and transmits signals and/or data to a computing and/or display device wirelessly. The DED may thus be connected through a communication system to an external electronic device having data storage, computing and/or display capabilities. The external computing and/or display device generally hosts client firmware and/or software and processing firmware and/or software for processing, analyzing, communicating and/or displaying data. It will be appreciated that the division of functions and processing, such as data processing, analysis, communications and display functions as between the DED and the external computing and/or display device may vary depending on many factors and is, to at least some extent, discretionary.

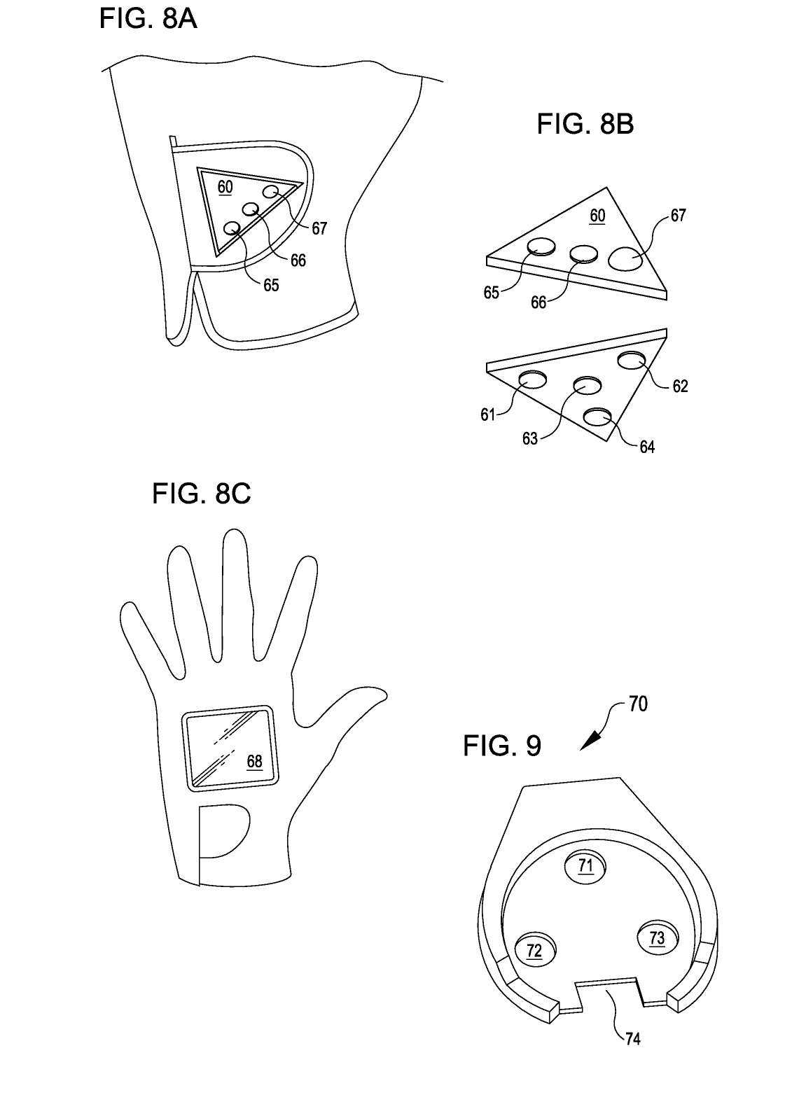

FIGS. 8A, 8B, 8C and 9 illustrate exemplary embodiments of DEDs and detachable placement of a DED on a glove. FIGS. 8A and 8B schematically illustrate a DED housing 60 having a triangular configuration with a plurality of signal receiving contacts 61, 62, 63, 64 exposed on one surface (See, FIG. 8B) and a plurality of visual indicators 65, 66, 67 (e.g., LEDs) on another exposed surface of the DED housing. The DED housing may be securely and removably received in a pocket (not shown) provided on the dorsal side of the glove, which in the illustrated embodiment is positioned on a glove adjustment tab. The DED may be configured to conveniently slide into the mating pocket, with signal receiving contacts engaging and electrically coupling to signal transfer contacts terminating on the glove tab for signal transfer, energy transfer, and the like. The visual indicators are preferable exposed for viewing or viewable through a transparent window in the pocket. Additional sensors, such as one or more accelerometer(s), gyroscopes, location sensors (e.g., GPS), optical or audio sensors, or the like may be provided in the DED.

FIG. 9 schematically illustrates a teardrop-shaped housing component 70 configured to be attached to a substrate (e.g., glove) for positioning and mating with a complementary DED. In this embodiment, housing 70 may be mounted or mountable to the substrate for mechanically (or magnetically or otherwise) facilitating positioning and retaining a mating DED in the housing, and for alignment of signal receipt and signal transfer terminals to facilitate operation of the sensing system. In the embodiment shown in FIG. 9, signal transfer and receipt terminals may be located in the area of the cavities 71, 72, 73 in the central region of the housing, and one or more additional signal/transfer terminals may be located in the region of lower cutout or receptacle 74 of the housing.

In some embodiments, as schematically illustrated in FIG. 8C, a display (68) capable of displaying content in addition to visual indicators (e.g., LEDs) may be provided and mounted to or integrated in a glove. The display may provide user-specific feedback relating to the sensed parameters and/or movement of the glove (and the user's hand) in space. When such a display is provided in connection with a golf glove, for example, the display may show the user's hand grip position and force, as sensed by sensors positioned in the glove, and the display may additionally show the motion of the user's hand through space as a swing is initiated and completed. In some embodiments, the display may additionally show, or extrapolate, the motion of an implement in the user's hand, such as a golf club head, and display the motion of the golf club head.

Additional and different content may be displayed on a display component associated with gloves having other sensing systems. In some embodiments, a display associated with a glove may be provided as an interactive display, such as a touch screen display. When an interactive (e.g., capacitive) touch screen display is provided, glove fingertips having conductive elements may be provided to use with and operate the capacitive display. A variety of glove types, such as sports gloves, outdoor gloves, motor sports gloves, ski gloves, and the like, may be provided with interactive or non-interactive display elements having rigid, curved or flexible components.

Sensor systems enabled in gloves, as described herein, may be used in combination with one or more additional sensor systems mounted for detection of movement and orientation on a glove, on an object gripped by a user (e.g., a golf club), on an auxiliary device attachable to a glove or a golf club, or on a garment, band, or the like wearable by the user. Suitable sensors include (single and/or multi-axis) accelerometer(s) for detecting magnitude and/or direction of the proper acceleration (or g-force), for sensing orientation, coordinate acceleration, vibration, shock, and the like; gyroscopes for detecting and monitoring orientation, angular momentum, and the like; camera(s) or other types of optical sensors and detectors; audio sensors and detectors for detecting sounds, vibrations, and the like, and other types of sensors. Sensors for detecting ambient conditions, such as temperature, humidity, light conditions, barometric pressure, and the like, may also be provided.

In some embodiments, client software and communications systems are hosted on the external computing device (e.g., a computer or a mobile device such as a tablet or smartphone), and provide feedback to and interact with the user, communicating through an Internet connection via web services, to push collected data and retrieve processed data from the service and display (or otherwise communicate) it to the user. The client software may comprise a set of applications that can run on multiple platforms (not limited to personal computers, tablets, smartphones) and sub-components (diagnostics, troubleshooting, data collecting, snap and match, shopping) to deliver a rich and complete user experience.

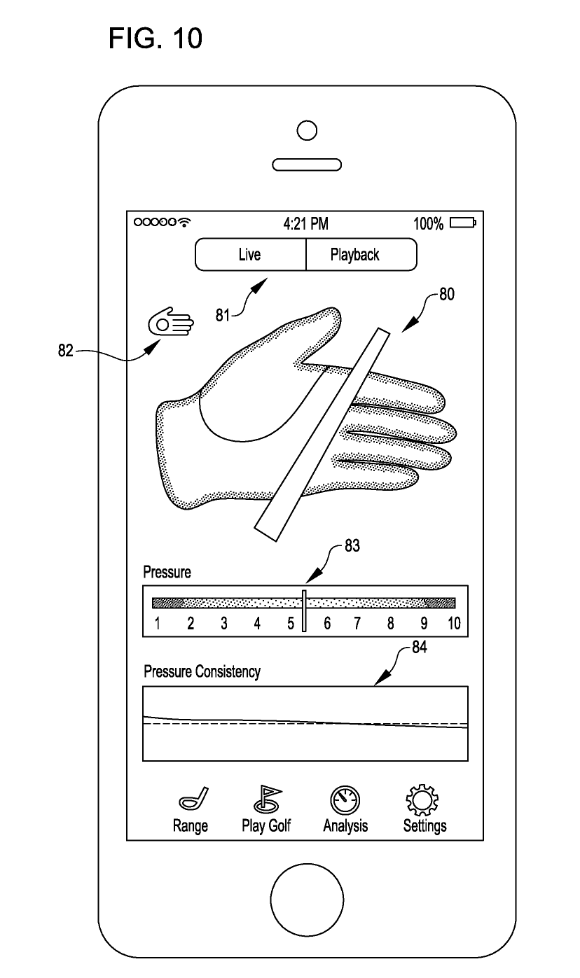

FIG. 10 illustrates a simple user interface displayed on a smart phone (but also displayable on a different display device, including a display device associated with a glove or another wearable component) providing a graphic presentation of a golf club grip in a user's hand, as detected using a sensor-enabled glove as described herein. The user interface has “live” and “playback” options (81) presented as a toggle switch for data presentation, and has an icon (82) confirming that the glove and DED are connected and functioning. A graphic image 80 showing the position of the golf club grip in the user's hand is presented. If the user's grip position is detected as being less than optimal, an alert may be provided as a visual or audio or otherwise sensible alert. Gripping pressure may be mapped on the hand graphic at the sensor locations using different colors to indicate different gripping pressure levels. Different types of gripping pressure indications may additionally or alternative be provided, such as the pressure graphic 83 illustrated. If the grip pressure is detected as being too high or too low, or inappropriately distributed, an alert may be provided as a visual or audio or otherwise sensible alert. Consistency in hand position on the golf club grip and gripping pressure over time (e.g., during a golf swing) may be assessed and reported to the user in a pressure consistency graphic 84. A variety of navigational icons may also be presented, as shown in the lower portion of the user interface.

Additional data may be collected and displayed, as described. Data collected from one or more sensors, such as a multi-axis accelerometer, can be analyzed and provide information relating to the user's dynamic swing motion, and feedback, such as 3D displays and analytical information relating to the 3D swing plane of the hands and handheld object (e.g., golf club) can be provided. Data collected from one or more sensor(s), such as gyroscope(s), can be analyzed and provide information relating to hand and object orientation prior to, during and following movement of the handheld object for monitoring orientation prior to, during and following a user's putting motion, swinging motion, racket stroke, and the like. The use of audio sensor(s) (e.g., a microphone) provided in a sensing system as described herein, or provided in another user device (e.g., smartphone, computing device, camera, etc.) may provide a reference time point for when the ball is hit and may be integrated with other sensed signals to provide a high level of analytical feedback relating to the user's hand position, grip pressure, hand and object motion, and the like, prior to, during and following motion, activity, etc.

For some applications, server software components that apply crowdsourcing logic and/or machine learning technologies may be implemented to identify, profile, and cluster user data. The data may be stored in a database and may be continuously or intermittently updated with incoming user supplied and/or sensor supplied data. An optional software component that provides image and pattern recognition capabilities may also be implemented. This feature may allow a user to input data (e.g. images, external data accessed from databases, etc.) without entering any text input.

Sensor-enabled gloves and related electronics (e.g., DED) and analytical tools (apps, phones, computing and display devices, etc.), as described herein, may be beneficially used in a wide range of applications. Sensor-enabled gloves and related electronics may be advantageously used, for example, in sports applications, including gloves for racket sports (e.g., tennis, badminton, squash, racketball, etc.) and handball, gloves for baseball, and particularly batting gloves, gloves for lacrosse, soccer goalies, boxing and other types of martial arts, weightlifting, cycling, football, skiing, and the like. Sensor-enabled gloves may also be adapted for use as driving gloves, motorcycle gloves, gloves for occupational activities, such as safety work gloves, work gloves, construction gloves, landscaping gloves, factory and transport workers' gloves, mechanic's gloves, and the like. Sensor-enabled gloves as described herein may also be designed for use in electronic gaming applications, and for sensing hand position, grip pressure, hand orientation and/or motion, or the like, during electronic game playing. Military applications for sensor-enabled gloves as described herein are also contemplated. It will be appreciated that the configurations, orientations and types of sensors, and the analytical feedback provided, may differ in these different applications.

In the description provided herein, the term “about” means+/−20% of the indicated value or range unless otherwise indicated. The terms “a” and “an,” as used herein, refer to one or more of the enumerated components or items. The use of alternative language (e.g., “or”) will be understood to mean either one, both or any combination of the alternatives, unless otherwise expressly indicated. The terms “include” and “comprise” are used interchangeably and both of those terms, and variants thereof, are intended to be construed as being non-limiting.

Although specific embodiments have been illustrated and described with reference to the sensors, leads, traces and terminals, described herein, as well as different types of DEDs, data collection, processing and analysis techniques, it will be appreciated that the same or similar types of sensing systems may be adapted for use in other types of applications, in connection with other types of garments, and in non-garment applications. Similar types of pliable e-textile sensors having resistive and/or conductive properties may be applied to or associated with a wide variety of non-conductive underlying flexible substrate materials, including woven and non-woven materials, and incorporated in a variety of sensor systems. Additional exemplary systems are described below, and are non-limiting.

While the present invention has been described above with reference to the accompanying drawings in which particular embodiments are shown and explained, it is to be understood that persons skilled in the art may modify the embodiments described herein without departing from the spirit and broad scope of the invention. Accordingly, the descriptions provided above are considered as being illustrative and exemplary of specific structures, aspects and features within the broad scope of the present invention and not as limiting the scope of the invention.

1. A sensing system implemented in a glove, comprising: at least two pressure sensors associated with a surface of the glove; at least two electrically conductive leads extending from each sensor; and at least one electrically conductive trace connected to each of the conductive leads, wherein each of the at least two sensors is positioned for contacting portions of an individual's hand during use and for detecting the position and/or magnitude of force, pressure and/or shear exerted against the sensor.

2. The sensing system of claim 1, wherein the at least two sensors are fabricated from a resistive e-textile material.

3. (canceled)

4. The sensing system of claim 1, wherein at least one sensor is located to contact a palmer location below the metacarpophalangeal joint (MCP) V when the glove is worn by a user.

5. (canceled)

6. The sensing system of claim 5, wherein at least one sensor is located to contact the metacarpophalangeal joint (MCP) II when the glove is worn by a user.

7. The sensing system of claim 1, wherein at least one of the sensors is partially or fully enclosed in a flexible barrier material.

8. (canceled)

9. (canceled)

10. (canceled)

11. The sensing system of claim 1, additionally comprising at least one additional sensor associated with the glove that provides data relating to at least one of the following: temperature, moisture, humidity, stress, strain, galvanic skin response, heart rate, respiratory rate, blood pressure, blood oxygen saturation, blood flow, local gas content, bacterial content, multi-axis acceleration, orientation and position.

12. (canceled)

13. The sensing system of claim 1, additionally comprising a galvanic skin response sensor associated with the glove.

14. (canceled)

15. The sensing system of claim 1, additionally comprising an electronic display associated with the glove.

16. The sensing system of claim 15, wherein the electronic display is an interactive display capable of receiving input from the user and displaying content to the user.

17. (canceled)

18. A glove having at least two pressure sensors located in a palmer region of the glove, each of the at least two pressure sensors being connected to at least two leads, each of the at least two leads being connected to at least one conductive trace, and each of the conductive traces being electrically connected to at least one signal transfer terminal positioned and configured for mating with a signal receiving terminal located on an associated electronic device.

19. (canceled)

20. The glove of claim 18, wherein at least one of the pressure sensors is located to contact the metacarpophalangeal joint (MCP) II when the glove is worn by a user.

21. The glove of claim 18, wherein at least one of the pressure sensors is located to contact a palmer location below the metacarpophalangeal joint (MCP) V when the glove is worn by a user.

22. (canceled)

23. (canceled)

24. (canceled)

25. The glove of claim 18, wherein first and second pressure sensors are located to contact a palmer region when the glove is worn by a user and a third pressure sensor is located to contact a finger when the glove is worn by a user.

26. The glove of claim 18, additionally comprising a plurality of signal receipt terminals located in a region near a base of the glove.

27. The glove of claim 18, additionally comprising a plurality of signal receipt terminals located in a region near an adjustable closure tab of the glove.

28. The glove of claim 18, additionally comprising a dedicated electronic device having signal receipt terminals that mate with signal transfer terminals located on the glove.

29. The glove and dedicated electronic device of claim 28, wherein the signal receipt and signal transfer terminals mate mechanically and magnetically.

30. (canceled)

31. The glove and dedicated electronic device of claim 28, wherein the dedicated electronic device additionally comprises at least one sensor for detecting at least one of hand position, hand orientation and hand motion.

32. (canceled)

33. The glove and dedicated electronic device of claim 28, wherein the dedicated electronic device communicates wirelessly with an external computing and/or display device.

34. The glove and dedicated electronic device of claim 28, wherein the dedicated electronic device additionally comprises a sensor for detecting at least one of the following: magnitude and/or direction of acceleration; coordinate acceleration; vibration; shock; orientation; angular momentum; optical data sounds; ambient temperature, ambient humidity; ambient light conditions; and barometric pressure.

35. The glove and dedicated electronic device of claim 28, wherein a combination of sensors provides signals for determining, at one or more time points: a user's hand position when gripping a handheld object; a user's gripping pressure when gripping the handheld object, and the orientation of the user's hands and of a portion of the handheld object.

36. (canceled)

37. The glove and dedicated electronic device of claim 28, wherein the combination of sensors additionally provides signals for determining and displaying a geo-referenced location of the user during movement.

38. The glove and dedicated electronic device of claim 28, wherein the combination of sensors additionally provides signals for determining and displaying a time point during movement when an object is struck.
